(19) United States
(12) Patent Application Publication
(10) Pub. No.: US 2020/0275888 A1
(43) Pub. Date: Sep. 3, 2020

(54) BIOLOGICAL INFORMATION PRESENTATION SYSTEM AND TRAINING METHOD

(71) Applicant: TOYOBO CO., LTD., Osaka (JP)

(72) Inventors: Yoko KOMATSU, Shiga (JP); Euichul KWON, Shiga (JP); Shota MORIMOTO, Shiga (JP); Yusuke SHIMIZU, Shiga (JP)

(73) Assignee: TOYOBO CO., LTD., Osaka (JP)

(21) Appl. No.: 16/649,775

(22) PCT Filed: Sep. 25, 2018
(86) PCT No.: PCT/JP2018/035331

§ 371 Date: Mar. 23, 2020

(30) Foreign Application Priority Data
Sep. 26, 2017 (JP) 2017-184991

(51) Int. Cl. A61B 5/00; A61B 5/0408; A61B 5/0478; A61B 5/0492; A61B 5/08; A61B 5/16

(52) U.S. Cl. CPC A61B 5/6804; A61B 5/0408; A61B 5/0478; A61B 5/0492; A61B 5/0809; A61B 2562/0217; A61B 5/486; A61B 2562/0209; A61B 2503/10; A61B 2503/22; A61B 2503/12; A61B 5/165

(57) ABSTRACT

The purpose of the present invention is to provide an efficient training system by obtaining biological information, converting the biological information into information indicating a mental state, a physiological state, and giving a feedback to a subject without giving a sense of discomfort to the subject. Provided is a biological information presentation system having a garment-type biological information measuring apparatus that includes at least fabric having a 20% elongation stress of 20 N or less, the garment pressure is 0.1 kPa or more and 1.5 kPa or less, and the skin contact-type electrode is provided at a portion in which a garment pressure is 0.3 kPa or more. The garment-type biological information measuring apparatus is worn on the subject, physiological information and mental information are obtained from the obtained biological information, results thereof are presented to a terminal device, and actions are presented on the basis of the results, thereby achieving efficient training.

### TECHNICAL FIELD

The present invention relates to a system that converts biological information obtained by using a garment-type biological information measuring apparatus, that is, a wearable sensing device, into information indicating a mental state and/or a physiological state of a wearer, and presents the information to the wearer or a third party in real time, and more specifically, relates to a training method of performing training of a machine operation, sport, or an instrumental performance using the system.

### BACKGROUND ART

In training for an athlete, a martial artist, and the like, various work training, and self-development, an attempt to grasp a mental state and utilize it for training has been made.

Patent Document 1 discloses that satisfaction of a worker who is working on a machine is measured with brain waves, a state of the satisfaction is fed back to the worker, and further the feedback is also applied to the machine. This method is a method for evaluating satisfaction based on physiological data such as brain waves, but a sense such as satisfaction is a complex sense, and thus a specific mental state cannot be evaluated. For example, it is impossible to grasp a specific mental state such as whether the satisfaction has been generated by relaxation, sleepiness, or awakening.

In addition, important physiological information can be obtained from brain waves, but it is difficult to sufficiently evaluate a specific mental state with only the brain waves. In addition, Patent Document 1 discloses a system that can provide a feedback of a satisfaction state to the worker, but the system is not a training system that the worker who has been recognized the satisfaction state can change his/her own state by his/her own intention, grasp the effect, and control his/her own mental state.

Patent Document 2 discloses a system for determining a psychological state by grasping a correspondence relation between an operation speed (reaction speed) of a game, a psychological state, and physiological data of an adrenocorticotropic hormone (ACTH). In general, a stress is evaluated with ACTH. However, in Patent Document 2, it is not clear and not specific what psychological state is to be grasped. In addition, Patent Document 2 discloses a system for notifying a user of a determination result of a psychological state, but the system is not a training system that a user can control his/her own mental state himself/herself by receiving the determination result to change the psychological state and can confirm the result. Since ACTH is not an index that represents the result in real time, it is considered to be technically problematic. In addition, it is difficult to evaluate a specific mental state such as relaxation, awakening, sleepiness, or tension with ACTH.

Patent Document 3 discloses a system that detects brain waves, evaluates comfort based on the brain wave information, and controls a device based on a determination result. Patent Document 3 discloses that relaxation and awakening can be evaluated with brain waves, but in a case where electrocardiographic data is added, the accuracy is further increased. In addition, Patent Document 3 discloses a system that receives a result obtained by evaluating comfort and controls a device, but the system is not a training system that a user changes him/her own mental state himself/herself and grasps the effect.

Patent Document 4 discloses a device that images a movement of a line of vision of a driver with a CCD camera, determines a distraction degree of a vehicle occupant from the result of the image analysis, and outputs a warning that the distraction degree is high. The mental state is a specific content such as “distraction degree”, but is not physiological data and is data obtained by an evaluation method by imaging facial expression. In addition, the determination result is fed back to a machine, but a human does not attempt to control his/her own mental state himself/herself by receiving the result.

Patent Document 5 discloses a system that measures physiological information of an infant, estimates a psychological state, and thus can make a notification by an emergency notification unit in a case where it is determined that an abnormality happened to the infant. A pulse is measured as a physiological value, such that a case in which an infant is crying because the infant is sleepy can be determined, or whether an infant is in a dangerous state such as lying on their face down can be determined. The determination result can be notified, but a human does not attempt to control himself/herself by receiving the result.

As disclosed in Patent Document 5, a system that measures physiological information, estimates a psychological state, displays an evaluation result, and further gives a feedback to a machine, is configured in the related art. However, it is not a system that continuously presents a mental state to a subject in real time as the time is elapsed. Therefore, it is impossible to perform training that a subject controls his/her own mental state himself/herself by receiving a presented mental state obtained by evaluating a physiological measurement value.

In addition, a physiological measurement value is often evaluated by a single measurement value such as only a brain wave, only an adrenocorticotropic hormone (ACTH), and only a pulse. It is a matter of course that a psychological state can be estimated to some degree with a single measurement value, but a plurality of indices are preferably used in order to more accurately estimate a specific mental state. Since the pulse represents an autonomic nerve activity and the brain wave represents a central nerve activity, a specific mental state can be evaluated, but it is difficult to evaluate a complex and specific mental state. In addition, since the system is not intended for training, the system is terminated at a stage at which the result is displayed, that is, it is not a system that a subject performs training by receiving the result and learns the effect himself/herself. In addition, in many documents, it is disclosed that a feedback is applied to a machine, but a system that instructs a subject to do a subsequent specific action is not provided.

Patent Document 6 discloses a mental training system that grasps a mental state using both brain wave information and electrocardiographic information, and performs a mental training by giving a feedback to a subject. By using both brain wave information and electrocardiographic information, it is possible to accurately grasp a mental state. However, since an electrocardiographic signal is an mV level, while brain wave information is a weak signal of about pV, even though a signal can be obtained in a laboratory, it is extremely difficult to measure a signal in outdoors, especially in noisy environments such as an intense exercise site or an actual work site.

In addition, even in the electrocardiographic measurement, it is difficult to play an intense sport or to perform actual work while mounting electrocardiograph used in a medical examination, and furthermore, wearing such a measuring apparatus on a body may be stressful itself. Therefore, a difference between an actual state and a measured state is generated when mental information is grasped in a state in which such an apparatus is mounted. As a result, it is difficult to be used for an appropriate training.

### PRIOR ART DOCUMENTS

### Patent Documents

Patent Document 1: JP-A-10-262942

Patent Document 2: JP-A-2004-267296

Patent Document 3: JP-A-8-71050

Patent Document 4: JP-A-8-290725

Patent Document 5: JP-A-2004-181218

Patent Document 6: Japanese Patent No. 4844523

### SUMMARY OF THE INVENTION

### Problems to be Solved by the Invention

The present invention has been made in view of such circumstances, and an object of the present invention is to provide a training system that grasps a mental state and gives a feedback to utilize the feedback in training, the mental state being grasped using a garment-type biological information measuring apparatus that is easily wearable and does not give a sense of discomfort to a wearer at the time of wearing.

### Solutions to the Problems

As a result of intensive studies in order to achieve the above object, the inventors of the present invention have developed a garment-type biological information measuring apparatus (sensing wear or wearable smart device) that is easily wearable and does not give a sense of discomfort to a wearer at the time of wearing, and have invented a biological information presentation system using the garment-type biological information measuring apparatus and a training method using the system.

That is, the present invention has the following constitution.

[1] A biological information presentation system that converts biological information obtained by using a garment-type biological information measuring apparatus into information indicating a mental state and/or a physiological state of a wearer, and presents the information to the wearer and/or a third party in real time,

wherein the garment-type biological information measuring apparatus includes at least fabric having a 20% elongation stress of 20 N or less,

a garment pressure is 0.1 kPa or more and 1.5 kPa or less, and

a skin contact-type electrode is provided at a portion in which a garment pressure is 0.3 kPa or more.

[2] The biological information presentation system according to [1], wherein the skin contact-type electrode is an electrode including conductive fabric.
[3] The biological information presentation system according to [1], wherein the skin contact-type electrode is an electrode including a stretchable conductive composition.
[4] The biological information presentation system according to [1], wherein the skin contact-type electrode is an electrode including a conductive gel.
[5] A training method of performing training of a work using the biological information presentation system according to any one of [1] to [4].
[6] The training method according to [5], wherein the work is sport.
[7] The training method according to [5], wherein the work is an instrumental performance.
[8] The training method according to [5], wherein the work is a machine operation.

Further, the present invention preferably has the following configuration.

[9] The biological information presentation system according to any one of [1] to [4] and the training method according to any one of [5] to [8], wherein the biological information is electrocardiographic information.
[10] The biological information presentation system according to any one of [1] to [4] and the training method according to any one of [5] to [8], wherein the biological information is myoelectric distribution information.
[11] The biological information presentation system according to any one of [1] to [4] and the training method according to any one of [5] to [8], wherein the biological information is brain wave information.
[12] The biological information presentation system according to any one of [1] to [4] and the training method according to any one of [5] to [8], wherein the biological information is respiration information.
[13] The biological information presentation system according to any one of [1] to [4] and the training method according to any one of [5] to [8], wherein at least two or more selected from electrocardiographic information, myoelectric distribution information, brain wave information, and respiration information are used as the biological information.
[14] The biological information presentation system and the training method according to any one of [9] to [13], wherein an SN ratio of the biological information is 10 dB or more.

The garment-type biological information measuring apparatus of the present invention preferably includes a wire including a stretchable conductive material. When the stretchable conductive material is appropriately used, in a case where at least fabric having a 20% elongation stress of 20 N or less is used, it is easy to configure a garment-type biological information measuring apparatus having a garment pressure of 0.1 kPa or more and 1.5 kPa or less, such that a portion having a garment pressure of 0.3 kPa or more can be implemented. In addition, as the stretchable conductive material, a layer including a stretchable conductive composition (film, sheet, or membrane), conductive yarn stitched into fabric in zigzag, conductive yarn incorporated in knit fabric, wire or metal foil patterns arranged with redundancy, and the like can be used.

### Effects of the Invention

The garment-type biological information measuring apparatus of the present invention has an appropriate garment pressure, such that the wearer can wear the apparatus without a sense of discomfort. Further, the skin contact-type electrode is adequately arranged at a portion in which a contact pressure is applied, in order to detect biological information, such that a signal is surely obtained and the wearer does not feel peculiar discomfort caused by an electrode portion. As a result, an operation such as sport or work can be naturally performed while wearing the biological information measuring apparatus. Further, the biological information measuring apparatus is in relatively closely contact with a body, such that the body functions as a buffer for noise itself, thereby improving an SN ratio.

The biological information obtained by this way is analyzed according to a conventional method and converted into information indicating a physiological state, and the information can be presented to a wearer or a supervisor. Here, physiological information is construed as comprehensive information about human mind and body including mental information in a broad sense.

The physiological information is appropriately fed back to the wearer, such that an efficient and adequate training can be performed.

### BRIEF DESCRIPTION OF THE DRAWINGS

FIG. 1 is a block diagram of a biological information presentation apparatus of the present invention.

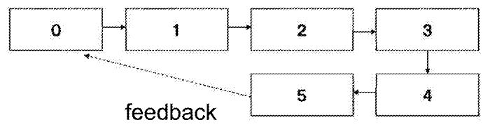

### MODE FOR CARRYING OUT THE INVENTION

A detection unit 1 that detects electrocardiographic information, brain wave information, myoelectric distribution information, and respiration information as biological information in a biological information measuring apparatus of the present invention will be described.

The electrocardiographic information, brain wave information, and myoelectric distribution information can be obtained as an electric signal. It is preferable that the information is obtained by performing a voltage measurement over time through a biological contact type electrode. In addition, an input impedance of a voltage measuring unit is 100 kΩ or more, preferably 300 kJ or more, and more preferably 1 MΩ or more. An upper limit thereof is not particularly limited.

The respiration information can be obtained from a shape change of a human body and a wind speed change near the mouth or the nose. In the present invention, a method of obtaining respiration information from a peripheral change of the human body is preferable in terms of reducing a sense of discomfort against a wearer. Such a peripheral change is finally converted into an electric signal by a sensor.

A garment which is a base of the garment-type biological information measuring apparatus of the present invention is made of fabric having a 20% elongation stress of 20 N or less. In addition, a garment pressure is set to be 0.1 kPa or more and 1.5 kPa or less. The garment pressure is premised on a person who has a standard shape, but it is preferable that a shape of a subject and a size of a garment are adjusted so that a garment pressure falls within an allowable range.

In the present invention, a skin contact-type electrode is arranged at a portion in which the garment pressure is 0.3 kPa or more. In general, the skin contact-type electrode is often brought into contact with a body at a pressure higher than necessary in order to achieve reliable contact. However, with such an arrangement, the sense of discomfort of the subject cannot be removed, and effective biological information cannot be obtained.

In the present invention, instead of electrocardiographic information, pulse wave information obtained by capturing a change in a blood flow rate rather than a biological potential can be used. The pulse wave can be measured at the wrist or fingers. In addition, a parameter relating to a blood pressure can be calculated from a difference between electrocardiographic information and pulse wave information at a position distant from the heart.

Although brain wave information is obtained by a medically prescribed method, in the present invention, fragmentary information of brain wave information is sufficient, for example, information obtained by one point measurement of a front head Fz or a top head Cz is also sufficient. The brain wave is not sufficient to be used as biological information compared to other information, and is easily to be buried in noise at an actual work space. Accordingly, the brain wave is used as auxiliary data in the present invention.

The brain wave is a weak potential change with a certain rhythm related to an electrical activity of the brain, and is classified into a δ wave, a θ wave, an α wave, a β wave, and a γ wave according to a frequency. Various studies on a sleep brain wave, a brain wave indicating comfort, and the like have been conducted, but in the present invention, a brain wave is detected for evaluating sleepiness and awakening, and only an a wave may be detected.

Next, a signal processing unit 2 that processes a signal from the detection unit will be described. An R wave is detected from electrocardiographic information. The R wave is a wave having the largest amplitude among the waves of the electrocardiographic information. A time interval (RR interval) between an R wave and an immediately preceding R wave is calculated every second. The brain wave is subjected to an FFT treatment with data of 512 points for 5 seconds, and an a wave power spectrum is calculated every second. In addition, the inverse of the result is calculated. FFT may use either a Hanning window or a Hamming window. In this way, the result of the RR interval and the result of the inverse of the a wave power spectrum are calculated every second. A pulse wave may be measured by calculating a time interval between a pulse wave and a subsequent pulse wave, similar to the RR interval of electrocardiographic information.

Next, an evaluation unit 3 that receives the signal processing result and evaluates a mental state will be described. A subjective evaluation experiment on a mental state such as relaxation, tension, awakening (activity), or sleepiness was carried out and factor analysis of the results of a questionnaire survey was performed, and as a result, it was found that relaxation and tension are present at the same axes and awakening (activity) and sleepiness are present at the same axes. In addition, as a result of examining the correspondence between the mental state and a physiological measurement value, it was found that there are a favorable correspondence between a relaxation-tension axis and the RR interval of electrocardiographic information, and a favorable correspondence between an awakening (activity)-sleepiness axis and the inverse of the a wave power spectrum of the brain wave.

In the results, a mental state of “relaxed and awakened” indicates a physiological index in which the RR interval is large and the inverse of the a wave power spectrum is also large. On the other hand, a mental state of “relaxed and sleepy” indicates a physiological index in which the RR interval is large but the inverse of the a wave power spectrum is small.

In the present invention, in a case where an influence of a noise is large and it is difficult to detect an a wave from brain wave information, a sleepiness index obtained from pattern analysis of an R-R interval of electrocardiographic information may also be used.

In the present invention, since a biological information measuring environment that does not give a sense of discomfort to a subject is realized, it is possible to evaluate a mental state with only electrocardiographic information without using brain wave information.

Next, an evaluation result presentation unit 4 that continuously represents a mental evaluation result to a subject in real time as the time is elapsed will be described. A signal processing result obtained by calculating an RR interval and an inverse of an a wave power spectrum every second is displayed on a monitor screen. The RR interval of electrocardiographic information and the inverse of the a wave power spectrum are taken as an X axis and a Y axis, respectively, and a value is continuously plotted every second so that a change over time can be seen. In order to easily see the lapse of time, a method such as a method of changing a color density over time or a method of changing a color every minute is preferable. When training is performed for a long period of time, it is possible to display the results by further increasing a display interval rather than 1 second.

In a case where it is difficult to obtain brain wave information, a sleepiness level obtained from the electrocardiographic information may be plotted at the Y axis.

After starting training, plotting is initiated at the center. By doing so, the change can be displayed to be easily seen. Ranges of the X axis and Y axis may also be changed and displayed as the plotting is increased so that the ranges can be displayed using the entire screen. In a case where a tension-relaxation axis is taken as an X axis, and a sleepiness-awakening axis is taken as a Y axis using the RR interval data of electrocardiographic information, and for example, a measurement result is plotted every minute by using inverse data of the a wave power spectrum or sleepiness information obtained from electrocardiographic information, mental information can be visually presented. It should be noted that in the present invention the real time means that the result is displayed whenever the mental information evaluation result is obtained. In a case where the time is required for the evaluation calculation, it is allowed to be delayed by the time until the result is displayed.

Next, an action presentation unit 5 that presents a specific recommended action will be described. The relaxation-tension axis can be digitized by the RR interval of electrocardiographic information, but electrocardiogram can be changed by a respiration adjustment because electrocardiogram is controlled by the automatic nervous system. In a case where an operation is performed by a subject himself/herself, when the subject wants to more relax after confirming a mental state evaluation result, the subject selects “relaxation” of a desired mental selection screen on a monitor. In response to this, an instruction such as “Close your eyes and take a deep breath” is displayed on the monitor. In a case where the selection is made to be in a more tense state, an instruction such as “Deliberately take a quick breath” is displayed on the monitor.

In a case where the subject wants to be more awakened after confirming a mental state evaluation result, the subject selects “awakening” of the desired mental selection screen on the monitor. In response to this, an instruction such as “Close your eyes and think a pleasant plan such as hobby” is displayed on the monitor. In a case where the selection is made to sleep more, an instruction such as “Close your eyes and do not think about anything” is displayed on the monitor. A specific content of the instruction is not limited to the content described above. The content also includes an instruction of calisthenics or an instruction to eat food. In addition, in a case where the subject wants to maintain the mental state, the subject selects “No change” that represents that the subject wants to maintain the mental state. FIG. 3 illustrates an example of the display method, but the present invention is not limited thereto.

Hereinabove, the case where the operation is performed by the subject himself/herself by confirming the presentation result is described, but it is possible to perform an appropriate action presentation by confirming the presentation result by a supervisor or a trainer. By using the present system, an appropriate action presentation based on physiological data can be performed, such that higher quality training is performed.

As the skin contact-type electrode in the present invention, an electrode including conductive fabric can be used. The conductive fabric is woven fabric, nonwoven fabric, knit fabric, embroidery yarn, sewing yarn, and the like formed of fiber including at least conductive yarn.

The conductive yarn is preferably yarn having a resistance value of 100Ω or less per 1 cm of fiber length. The conductive yarn is a general term for conductive fiber, a fiber bundle of conductive fiber, twisted yarn, plaited yarn, spun yarn, and blended yarn obtained from fiber including conductive fiber, a fine metal wire obtained by finely stretching a metal wire, and a fine film obtained by cutting a film into a fine fiber shape.

Examples of the conductive fiber include chemical fiber or natural fiber coated with a metal, chemical fiber or natural fiber coated with a conductive metal oxide, chemical fiber or natural fiber coated with a carbon-based conductive material such as graphite, carbon, carbon nanotube, and graphene, and chemical fiber or natural fiber coated with a conductive polymer.

In addition, as the conductive fiber, for example, fiber obtained by spinning a polymer material including at least one conductive material selected from the group consisting of a metal, a conductive metal oxide, a carbon-based conductive material, and a conductive polymer, can be used.

As the fiber bundle of conductive fiber, for example, a fiber bundle obtained by charging and impregnating a fiber bundle formed of micro fiber or nanofiber of the conductive fiber with a conductive filler or a conductive polymer, can be used.

As the conductive yarn, twisted yarn, plaited yarn, spun yarn, and blended yarn obtained from fiber including the conductive fiber may also be used.

The conductive yarn also includes a fine metal wire obtained by finely stretching a metal wire.

An average diameter of each of the conductive fiber, the fiber bundle of conductive fiber, the twisted yarn, plaited yarn, spun yarn, and blended yarn obtained from fiber including the conductive fiber, or the fine metal wire is preferably 250 μm or less, more preferably 120 μm or less, still more preferably 80 μm or less, and particularly preferably 50 μm or less.

The conductive yarn also includes a fine film obtained by cutting a film into a fine fiber shape, the fine film refers to a fibrous film obtained by cutting a polymer film to a width of 800 μm or less, the polymer film being coated with at least one conductive material selected from the group consisting of a metal, a conductive metal oxide, a carbon-based conductive material, and a conductive polymer.

Among the conductive yarns, it is preferable to use chemical fiber coated with a metal, a fiber bundle of conductive fiber with which a conductive polymer is charged and impregnated, and conductive yarn including at least one selected from the group consisting of a fine metal wire having an average diameter of 50 μm or less.

Specific examples of the conductive fabric include a fiber structure obtained by embroidering conductive yarn on non-conductive cloth, a fiber structure obtained by impregnating non-conductive cloth with a conductive polymer-containing solution and performing drying, and a fiber structure obtained by performing impregnation with a solution containing a conductive filler and a binder resin, and performing drying. Among them, it is preferable to use a fiber structure obtained by impregnating non-conductive cloth with a conductive polymer-containing solution and performing drying.

As the conductive polymer, for example, a mixture containing poly(3,4-ethylenedioxythiophene) and polystyrene sulfonic acid can be preferably used.

As the fiber including conductive yarn, a synthetic fiber multifilament is preferable, and it is preferable that at least a part of the synthetic fiber multifilament is a fine filament having a fineness of less than 30 dtex, or a synthetic fiber multifilament having a fineness of more than 400 dtex and a single fiber fineness of 0.2 dtex or less.

In a case where the conductive fabric is textile formed of fiber including conductive yarn, or knit, a basis weight is preferably less than 50 g/m², whereby the conductive polymer can be prevented from falling off. In addition, the basis weight is preferably more than 300 g/m², whereby a sufficient conductivity can be secured.

As the skin contact electrode of the present invention, an electrode including a stretchable conductive composition can be used. The stretchable conductor layer has stretchability, and refers to a layer having a specific resistance of 1×10⁰ Ωcm or less. The stretchability means that 10% or more stretch can be repeated while maintaining conductivity. The single stretchable conductor layer preferably has 40% or more breaking elongation. The breaking elongation is more preferably 50% or more and still more preferably 80% or more.

A conductive paste is coated on a release sheet at a predetermined thickness, the release sheet is dried and then peeled off, and then a tensile test is performed, thereby measuring the breaking elongation.

A tensile modulus of the stretchable conductor layer is preferably 10 to 500 MPa.

An average thickness of the stretchable conductor layer is, for example, preferably 20 μm or more and preferably 50 μm or less. The average thickness of the stretchable conductor layer is more preferably 500 μm or less, still more preferably 250 μm or less, and particularly preferably 90 μm or less.

Hereinafter, a material that can form such a stretchable conductor layer may be referred to as a composition for a stretchable conductor layer. The stretchable conductor layer can be formed using, for example, a conductive paste as the composition for a stretchable conductor layer.

The conductive paste contains at least (i) a conductive particle, (ii) a flexible resin, and (iii) a solvent.

(i) Conductive Particle

The conductive particle refers to a particle having a specific resistance of 1×10⁻¹ Ωcm or less.

Examples of the particle having a specific resistance of 1×10⁻¹ Ωcm or less include a metal particle, an alloy particle, a carbon particle, a carbon nanotube particle, a doped semiconductor particle, a conductive polymer particle, and a hybrid particle.

Examples of the metal particle include a silver particle, a gold particle, a platinum particle, a palladium particle, a copper particle, a nickel particle, an aluminum particle, a zinc particle, a lead particle, and a tin particle.

Examples of the alloy particle include a brass particle, a bronze particle, a cupronickel particle, and a solder particle. Examples of the doped semiconductor particle include oxide of tin and a composite oxide of indium and tin. An example of the conductive polymer particle includes a particle including a mixture containing poly(3,4-ethylenedioxythiophene) and polystyrene sulfonic acid, or a polymer particle coated with a metal. Examples of the hybrid particle include a metal particle coated with a metal, a glass particle coated with a metal, and a ceramic particle coated with a metal. An example of the metal particle coated with a metal includes a silver coated copper particle.

An average particle diameter of the conductive particles is, for example, preferably 100 μm or less, more preferably 30 μm or less, and still more preferably 12 μm or less. A lower limit of the average particle diameter is not particularly limited, but is, for example, 0.08 μm or more.

The particle may be, for example, flake-shaped powder and may be amorphous agglomerated powder. For example, as the silver particle, a flake-shaped silver powder or amorphous agglomerated silver powder can be used.

An average particle diameter (50% D) of the flake-shaped powder measured by a dynamic light scattering method is, for example, preferably 0.5 to 20 μm. When the average particle diameter of the flake-shaped powder is less than 0.5 μm, the particles may not be in contact with each other, which may cause deterioration of conductivity. The average particle diameter of the flake-shaped powder is more preferably 3 μm or more and still more preferably 5 μm or more. However, when the average particle diameter of the flake-shaped powder exceeds 20 μm, it is difficult to form a fine wire. In addition, in a case where screen printing is performed, clogging may occur. The average particle diameter of the flake-shaped powder is more preferably 15 μm or less and still more preferably 12 μm or less.

An average particle diameter (50% D) of the amorphous agglomerated powder measured by a light confusion method is, for example, preferably 1 to 20 μm. When the average particle diameter of the amorphous agglomerated powder is less than 1 μm, the effect as agglomerated powder is lost, and the conductivity cannot thus be maintained. The average particle diameter of the flake-shaped powder is more preferably 3 μm or more and still more preferably 5 μm or more. However, when the average particle diameter of the amorphous agglomerated powder exceeds 20 μm, it is difficult for the powder to form a paste due to lowering of dispersibility in a solvent. The average particle diameter of the flake-shaped powder is more preferably 15 μm or less and still more preferably 12 μm or less.

(ii) Flexible Resin

As the flexible resin, a thermoplastic resin, a thermosetting resin, or rubber that has an elastic modulus of 1 Mpa or more and 1000 MPa or less can be used. It is preferable that rubber is used in order to implement stretchability of a film. The elastic modulus of the flexible resin is preferably 3 MPa or more, more preferably 10 MPa or more, and still more preferably 30 MPa or more. The elastic modulus of the flexible resin is preferably 600 MPa or less, more preferably 500 MPa or less, and still more preferably 300 MPa or less.

Examples of the thermoplastic resin can include polyethylene, polyvinyl chloride, polystyrene, polyvinyl acetate, polyurethane, an acrylic resin, polyamide, and polyester.

Examples of the thermosetting resin can include a phenolic resin, an epoxy resin, a melamine resin, and a silicone resin.

Examples of the rubber include urethane rubber, acrylic rubber, silicone rubber, butadiene rubber, nitrile group-containing rubber such as nitrile rubber or hydrogenated nitrile rubber, isoprene rubber, sulfide rubber, styrene butadiene rubber, butyl rubber, chloroprene rubber, chlorosulfonated polyethylene rubber, ethylene propylene rubber, and a vinylidene fluoride copolymer. Among them, nitrile group-containing rubber, chloroprene rubber, and chlorosulfonated polyethylene rubber are preferable, and nitrile group-containing rubber is particularly preferable.

The nitrile group-containing rubber is not particularly limited as long as it is rubber or an elastomer containing a nitrile group, for example, nitrile rubber and hydrogenated nitrile rubber are preferable. Nitrile rubber is a copolymer of butadiene and acrylonitrile, and when the amount of bonding acrylonitrile is increased, affinity with a metal is increased, but rubber elasticity contributing to stretchability is rather decreased. Therefore, the amount of bonding acrylonitrile in the acrylonitrile-butadiene copolymer rubber is preferably 18 to 50% by mass, and more preferably 40 to 50% by mass.

A content of the flexible resin is 7 to 35% by mass, more preferably 9% by mass or more, and still more preferably 12% by mass or more, but preferably 28% by mass or less, and more preferably 20% by mass or less, with respect to the total amount of the conductive particle and the flexible resin.

(iii) Solvent

The solvent is not particularly limited, and a well-known organic solvent or aqueous solvent can be used.

A surface of the electrode, that is, a side in contact with the skin of a wearer preferably has an electrode surface layer. Meanwhile, it is preferable that a base layer is provided at a boundary between the electrode and the cloth portion in order to increase insulating properties.

(Electrode Surface Layer)

Examples of the electrode surface layer include a noble metal plating layer, a metal layer that is hardly to be oxidized by a formation of a passive state, a corrosion resistant layer, a carbon layer, and a stretchable conductive layer, and these layers may be provided alone or may be provided by laminating two or more layers.

An example of the noble metal plating layer includes at least one layer selected from the group consisting of gold, silver, platinum, rhodium, and ruthenium.

An example of the metal layer that is hardly to be oxidized due to a formation of a passive state includes one layer selected from the group consisting of chromium, molybdenum, tungsten, and nickel.

An example of the corrosion resistant layer includes a layer including a monel alloy.

It is preferable that the carbon layer is formed by printing a carbon paste on the surface of the electrode.

It is preferable that the stretchable conductive layer includes a stretchable conductive composition containing a conductive filler and a flexible resin.

A conductive gel can be used for the skin contact-type electrode of the present invention. Here, the conductive gel may be construed as a gel electrode material used for a surface of the skin contact-type electrode used in medical instrument.

### EXAMPLES

### [Preparation of Conductive Paste]

Coatron KYU-1 manufactured by SANYO CHEMICAL, LTD. (glass transition temperature: −35° C.) was used as a binder, micro-diameter silver powder SPHO2J manufactured by MITSUI MINING & SMELTING CO., LTD. (average particle diameter: 1.2 μm) was used as a silver particle, Ketjen black EC600JD manufactured by Lion Specialty Chemicals Co., Ltd. was used as a carbon particle, and butyl carbitol acetate was used as a solvent, 10 parts by mass of the binder, 70 parts by mass of the silver particle, 1 part by mass of the carbon particle, and 19 parts by mass of the solvent were blended, thereby preparing a conductive paste for stretchable conductive formation. First, a binder resin was dissolved in a solvent of a half amount of a predetermined amount, premixing was performed by adding a metal particle and a carbon particle to the obtained solution, and then the solution was dispersed by a three-roll mill, thereby forming a paste.

The stretchable conductor layer (stretchable conductive sheet) had an initial specific resistance of 250 μΩ·cm and had stretchability at which conductivity is maintained even after 20% elongation was repeated 100 times, the stretchable conductor layer being obtained by performing screen-printing on the obtained paste for stretchable conductive formation at a thickness of 25 μm and drying the paste at 100° C. for 20 minutes.

### [Preparation of Stretchable Carbon Paste]

A carbon paste for an electrode protective layer was prepared according to the composition shown in Table 2.

40 parts by mass of a nitrile butadiene rubber resin having a glass transition temperature of −19° C., 20 parts by mass of Ketjen black EC300J manufactured by Lion Specialty Chemicals Co., Ltd., and 50 parts by mass of ethylene glycol monoethyl ether acetate as a solvent were pre-mixed and then dispersed by a three-roll mill, thereby obtaining a stretchable carbon paste.

A predetermined-shaped urethane sheet (corresponding to an insulating cover layer) from which an electrode portion and a connector part were cut out was temporarily bonded to a PET release sheet having a surface subjected to a treatment by a silicone-based release agent, a stretchable carbon paste was screen-printed at the electrode portion, a stretchable conductive paste was further printed in a predetermined pattern from the electrode portion and to the connector position, and then a double-sided hot melt sheet (corresponding to an insulating base layer) was laminated so as to cover the urethane sheet, thereby forming an electrode and a wire on the release sheet.

The obtained electrode and wire formed on the release sheet are stacked on fabric for a garment so that they are brought into contact with the double-sided hot melt sheet and are subjected to heating and pressing by a hot press, such that the electrode and the wire can be transferred to an electrode support portion together with the insulating base layer and the insulating cover layer.

### Example 1

An electrocardiographic information measurement electrode, which includes a stretchable conductive composition having a 20% elongation stress of 0.5 N, was attached to a chest portion of a sport shirt made of fabric having a 20% elongation stress of 7 N, and myoelectric distribution measurement electrodes including the same material that of the electrocardiographic information measurement electrode (8 electrodes were arranged around left and right arms, respectively) were attached around each arm, a stretchable capacitor having a 20% elongation stress of 1.2 N was arranged around a chest, an electrode potential and a capacity change of the stretchable capacitor were detected, and an electronic unit was attached so as to transmit data to a portable terminal, thereby manufacturing a garment-type biological information measuring apparatus capable of collectively measuring electrocardiographic information, myoelectric distribution information, and respiration information. When the obtained biological information measuring apparatus was worn on a subject, the maximum garment pressure was 0.6 kPa, a garment pressure of an electrocardiographic information measurement electrode arranged portion was 0.4 kPa, and a garment pressure of a myoelectric measurement electrode arranged portion was 0.6 kPa. In addition, an electrode including a stretchable conductive composition was produced by transferring the electrode and the wire obtained in advance and formed on the release sheet by a hot press. A thermometer, position information obtained by GPS, an acceleration sensor for XYZ axes are mounted on the electronic unit, and the electronic unit can also transmit information to the portable terminal.

A biological information presentation system was obtained by setting the information obtained from the garment-type biological information measuring apparatus to display the information on a tablet terminal as the portable terminal.

The garment-type biological information measuring apparatus was worn on a subject, and the tablet terminal was observed by a trainer. The subject is a biathlon athlete. Basic parameters such as electrocardiogram (heart rate), respiration, electromyogram, a joint angle, and body surface temperature of the subject in training were detected, an activity balance of sympathetic nerve and parasympathetic nerve, that is, a degree of tension and relaxation was calculated from frequency analysis of electrocardiographic waveform, and the training was performed while presenting the results to the tablet terminal and giving appropriate instructions from a trainer who is a supervisor.

In the case of shooting, a basic parameter such as a posture state (a motion of a joint and a tension state of muscle) or a psychological state (a degree of tension) was measured in real time during a series of operation from lifting of gun to firing, the measured data was analyzed by being combined with information of other measuring apparatuses (a motion of the muzzle of a gun, a score, a landing position, a motion of a line of vision, blinking, and the like), such that instructions could be presented so that a mental state and a physiological state of the subject were induced to the original concentration state.

In addition, in the case of cross country skiing, a degree of fatigue and a remaining amount of stamina were estimated from body movement (activity amount), an exercise amount, a breathing rate, and heart rate progress, and the estimated data was reflected to grasp a biological state during shooting.

It should be noted that the subject did not particularly appeal any discomfort about the garment-type biological information measuring apparatus, and the training could be naturally performed as usual.

### Example 2

An electrocardiographic information measurement electrode including conductive fabric and a stretchable capacitor having a 20% elongation stress of 1.2 N were arranged at an under-bust portion of a sport brassiere made of fabric having a 20% elongation stress of 5 N, an electrode potential and a capacity change of the stretchable capacitor were detected, and an electronic unit was attached so as to transmit data to a portable terminal, thereby manufacturing a garment-type biological information measuring apparatus capable of collectively measuring electrocardiographic information and respiration information. When the obtained biological information measuring apparatus was worn on a subject, the maximum garment pressure was 0.85 kPa. and a garment pressure of an electrocardiographic information measurement electrode arranged portion was 0.8 kPa.

A biological information presentation system was obtained by setting the information obtained from the garment-type biological information measuring apparatus to display the information on a tablet terminal as the portable terminal.

The subject who has been subjected to the training is a high jump athlete, the training being performed by allowing the subject to wear the garment-type biological information measuring apparatus and observing the tablet terminal by the trainer. Similarly to Example 1, various parameters of the subject were measured, the training in which the trainer instructed the subject to run-up at the time at which it was determined that the subject concentrated the mind and attempts were tried was repeated, and as a result, the game result was improved. It should be noted that the subject did not particularly appeal any discomfort about the garment-type biological information measuring apparatus, and the training could be naturally performed as usual.

### Example 3

An electrocardiographic information measurement electrode including a stretchable conductive composition having a 20% elongation stress of 3.5 N and a stretchable capacitor having a 20% elongation stress of 5.2 N were arranged at a waist portion of a brief made of fabric having a 20% elongation stress of 18N, an electrode potential and a capacity change of the stretchable capacitor were detected, and an electronic unit was attached so as to transmit data to a portable terminal, thereby manufacturing a garment-type biological information measuring apparatus capable of collectively measuring electrocardiographic information and respiration information. When the obtained biological information measuring apparatus was worn on a subject, the maximum garment pressure was 1.4 kPa, and a garment pressure of an electrocardiographic information measurement electrode arranged portion was 1.2 kPa.

A biological information presentation system was obtained by setting the information obtained from the garment-type biological information measuring apparatus to display the information on a tablet terminal as the portable terminal.

The subject who has been subjected to the training is a hammer thrower, the training being performed by allowing the subject to wear the garment-type biological information measuring apparatus and observing the tablet terminal by the trainer. Similarly to Example 1, various parameters of the subject were measured, the training in which the trainer instructed the subject to start throwing at the time at which it was determined that the subject concentrated the mind and attempts were tried was repeated, and as a result, the game result was improved. It should be noted that the subject did not particularly appeal any discomfort about the garment-type biological information measuring apparatus, and the training could be naturally performed as usual.

### INDUSTRIAL APPLICABILITY

As described above, the biological information presentation system using a garment for biological information measurement of the present invention can obtain biological information during training in a natural state without giving a sense of discomfort to a wearer, and an efficient training can be performed by using the system.

The present invention can be broadly applied regardless of men and women, and can be broadly used in various sport training such as ball game, gymnastics, swimming, shoot, Kyudo (Japanese art of archery), archery, throwing game, and martial arts, driving training such as a vehicle, a ship, an airplane, and a heavy machine for civil engineering, skill training such as wood working, iron working, metal carving, sewing working, dental technique, medical operation, and cooking, performance training such as wind instrument, string instrument, percussion instrument, and vocal musical, or art training such as chirography, calligraphy, sculpture, embroidery, and painting.

### DESCRIPTION OF REFERENCE SIGNS

0 Subject
1 Biological information detection unit
2 Signal processing unit
3 Evaluation unit
4 Evaluation result presentation unit
5 Action presentation unit

1. A biological information presentation system that converts biological information obtained by using a garment-type biological information measuring apparatus into information indicating a mental state and/or a physiological state of a wearer, and presents the information to the wearer and/or a third party in real time,
wherein the garment-type biological information measuring apparatus includes at least fabric having a 20% elongation stress of 20 N or less,
a garment pressure is 0.1 kPa or more and 1.5 kPa or less, and
a skin contact-type electrode is provided at a portion in which a garment pressure is 0.3 kPa or more.

2. The biological information presentation system according to claim 1, wherein the skin contact-type electrode is an electrode including conductive fabric.

3. The biological information presentation system according to claim 1, wherein the skin contact-type electrode is an electrode including a stretchable conductive composition.

4. The biological information presentation system according to claim 1, wherein the skin contact-type electrode is an electrode including a conductive gel.

5. A training method of performing training of a work using the biological information presentation system according to claim 1.

6. The training method according to claim 5, wherein the work is sport.

7. The training method according to claim 5, wherein the work is an instrumental performance.

8. The training method according to claim 5, wherein the work is a machine operation.
